(19) United States
(12) Patent Application Publication
(10) Pub. No.: US 2023/0364321 A1
(43) Pub. Date: Nov. 16, 2023

(54) Ultrasonic Aspirator Apparatus and Manipulation

(71) Applicant: Integra LifeSciences Enterprises, LLLP, Princeton, NJ (US)

(72) Inventors: Daniel J. Cotter, North Easton, MA (US); James Sheehan, North Reading, MA (US); Patrick Allen, Waltham, MA (US); Igor V. Kosenko, Boxborough, MA (US)

(21) Appl. No.: 18/315,360

(22) Filed: Jul. 13, 2023

Related U.S. Application Data
(60) Provisional application No. 63/340,873, filed on May 11, 2022.
(60) Provisional application No. 63/340,416, filed on May 10, 2022.

(51) Int. Cl. A61M 1/00; A61B 34/37; A61M 3/02

(52) U.S. Cl. CPC A61M 1/86; A61B 34/37; A61M 3/0279; A61B 2034/302

(57) ABSTRACT

A surgical apparatus includes a robotic system having a plurality of arm members. Each of the plurality of arm members is coupled to each adjacent arm member, and at least one of the plurality of arm members includes a first receiver and a second receiver. The apparatus further includes an aspirator having a handpiece, a connector extending from the handpiece, a horn coupled to the handpiece a flue coaxially disposed about the horn. The handpiece is received by the first receiver and the flue is received by the second receiver, such that the aspirator remains axially aligned relative to the at least one of the plurality of arm members when the robotic system is manipulated.

### TECHNICAL FIELD

The present disclosure generally relates to surgical apparatuses and methods of performing surgical operations, and more particularly, to robotically controlled surgical apparatuses and methods of performing surgical operations using robotic systems.

### BACKGROUND

Common surgical procedures, including minimally invasive surgeries (“MIS”) such as laparoscopy, are generally considered to be safe and effective procedures for treating a variety of ailments. However, these procedures often require at least one surgical assistant to help manipulate instruments during the procedure. Furthermore, these types of procedures can be physically demanding for a surgeon, particularly for procedures that involve a significant amount of time, precision, and control. Accordingly, a need exists for a surgical apparatus that may allow for a single individual to perform an operation while minimizing the risks commonly attributable to user error caused by fatigue and/or other issues with instrument manipulation.

### SUMMARY

In an embodiment, a surgical apparatus is disclosed. The surgical apparatus includes a robotic system having a plurality of arm members, with each of the plurality of arm members being coupled to each adjacent arm member via a joint, and at least one of the plurality of arm members including a first receiver and a second receiver. The surgical apparatus further includes an aspirator having a handpiece, a connector extending from the handpiece, a horn coupled to the handpiece via the connector, and a flue coaxially disposed about the horn and coupled to the handpiece via the connector. The handpiece of the aspirator is received by the first receiver of the at least one of the plurality of arm members and the flue of the aspirator is received by the second receiver of the at least one of the plurality of arm members, such that the aspirator remains axially aligned relative to the at least one of the plurality of arm members when the robotic system is manipulated.

In another embodiment, a surgical apparatus is disclosed. The surgical apparatus includes a robotic system having a plurality of arm members, with each of the plurality of arm members being coupled to each adjacent arm member via a joint, and at least one of the plurality of arm members including a first receiver and a second receiver. The surgical apparatus further includes an aspirator having a handpiece and a connector extending from the handpiece. The handpiece is received by the first receiver such that the connector extends from the first receiver. The aspirator further includes a horn coupled to the handpiece via the connector, the horn having a distal end, a proximal end, and a body extending therebetween, and a flue coaxially disposed about the horn and coupled to the handpiece via the connector, where the flue has a distal end, a proximal end, and a body extending therebetween, and the distal end of the flue is received by the second receiver such that the distal end of the horn extends beyond the distal end of the flue and the second receiver. The surgical apparatus further includes a console including an irrigation source that provides irrigation fluid to the aspirator via an irrigation tube, an aspiration source that provides suction to the aspirator via an aspiration tube, and a power source that provides power to the aspirator and the plurality of arm members. The connector further includes a vent for ventilating the irrigation tube to atmosphere when the irrigation source provides the irrigation fluid to the aspirator.

In yet another embodiment, a method of performing a surgical operation is disclosed. The method includes coupling an aspirator to a robotic system having a plurality of arm members, wherein at least one of the plurality of arm members includes a first receiver and a second receiver for receiving the aspirator; manipulating, via a console, a position and orientation of the plurality of arm members, such that the aspirator is moved to an entry position and an entry orientation relative a target site; inserting, using the plurality of arm members of the robotic system, the aspirator into the target site at the entry position and the entry orientation; activating, using the console, a horn of the aspirator such that the horn vibrates within the target site at a predetermined frequency; irrigating, via the console, the target site by supplying an irrigation fluid through an irrigation tube that extends from the console to the aspirator; aspirating, via the console, the target site by generating a vacuum within the aspirator using an aspiration tube that extends from the console to the aspirator; and removing, using the plurality of arm members of the robotic system, the aspirator from the target site.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a schematic view of a surgical apparatus including a robotic system and an aspirator, according to one or more embodiments shown and described herein;

FIG. 2 depicts a partially exploded perspective view of the aspirator of FIG. 1, according to one or more embodiments shown and described herein;

FIG. 3 depicts a partial cross-sectional view of the assembled aspirator of FIG. 2, according to one or more embodiments shown and described herein;

FIG. 4 depicts a schematic view of a console for controlling the surgical apparatus of FIG. 1, according to one or more embodiments shown and described herein; and

FIG. 5 depicts an illustrative flow diagram of a method of performing a surgical operation, according to one or more embodiments shown and described herein.

### DETAILED DESCRIPTION

Embodiments disclosed herein relate to surgical apparatuses and methods of performing surgical operations. More specifically, the present disclosure relates to a surgical apparatus that includes a robotic system and an aspirator, such as an ultrasonic surgical aspirator, which is coupled to the robotic system, such that the robotic system may manipulate the aspirator during the course of a surgical procedure. In the embodiments described herein, the aspirator may include a horn, such as an ultrasonic horn, a flue disposed coaxially about the horn, and a handpiece for securing the flue in place about the horn. The aspirator may further include an aspiration tube, an irrigation tube, and a power line that are used to operate the aspirator during a surgical procedure.

The robotic system described herein further includes a plurality of arm members that may be connected via a plurality of joints, such that the plurality of arm members are capable of rotating and/or moving through a plurality of planes in order to adjust the position of the aspirator during the surgical procedure. Furthermore, the robotic system may include a first receiver, such as a handpiece receiver, and a second receiver, such as a flue receiver, which may be used for securing the aspirator to the robotic system. In these embodiments, the first receiver may include at least one first receiver port, which may be axially aligned with an at least one second receiver port located within the second receiver. The handpiece of the aspirator may be coupled to the first receiver of the robotic system, such that a connector formed on the handpiece may extend through the at least one first receiver port of the first receiver and engage a proximal end of the flue and the horn. The aspirator may extend through and beyond the at least one second receiver port, such that the distal end of the flue is received by the second receiver and the horn extends through the second receiver, thereby allowing the horn to be inserted into a target site (e.g., a patient, etc.). In these embodiments, the first receiver and the second receiver may align the aspirator in a particular orientation, and may ensure that the aspirator remains in alignment throughout the surgical procedure.

As has been noted herein, the disclosed surgical apparatus aims to simplify a variety of minimally invasive surgeries (“MIS”), including laparoscopy, in order to make procedures more accessible to residents, fellows, surgeons, and other individuals trained to perform said procedures. For example, an obstacle in performing current laparoscopic procedures is the ability of a surgeon and/or other individual to provide an ultrasonic aspirator with stability (e.g, positional and/or rotational stability) and to manage retraction and positioning of tissues around the target site throughout the course of the procedure. During current procedures, an unstable aspirator (or other surgical instrument) may result in trauma, morbidity, blood loss, and other surgical complications. Furthermore, multiple individuals are often needed to control various functions of an aspirator during a laparoscopic procedure. For example, it may be necessary to provide ultrasonic aspiration and electrosurgical coagulation to a target site simultaneously during the surgical procedure. Accordingly, traditional laparoscopy procedures require multiple trained individuals to control the various functions of the ultrasonic aspirator and to ensure that the aspirator remains stable throughout the duration of the procedure.

In contrast, the disclosed surgical apparatus eliminates the need for multiple users by providing a robotic system for manipulating the ultrasonic aspirator. In these embodiments, the robotic system may be utilized to position and/or stabilize the ultrasonic aspirator while a single individual (e.g., surgeon) performs the laparoscopic procedure. Additionally, the robotic system may allow for a user to control multiple functions of the aspirator using a single control element. As will be described in additional detail herein, a user performing the procedure may be capable of controlling the robotic system throughout the course of the procedure (e.g., via foot pedal, etc.), such that the laparoscopy may be performed by the single user.

As provided herein, the terms “ultrasonic horn,” “ultrasonic tip,” “ultrasonic aspirating tip,” “ultrasonic surgical aspirating tip,” “aspirating tip,” “ultrasonic surgical tip,” “surgical tip” and “tip” may be used interchangeably. Additionally, it should be further appreciated that the term “ultrasonic” may further include radio frequency (“RF”). Similarly, the terms “flue,” “irrigation flue,” “sleeve,” “irrigation manifold” and “manifold” may be used herein interchangeably. The terms “tip extender” and “horn extender” may also be used herein interchangeably.

As defined herein, the term “non-disposable” is used to refer to a device that is intended for use on multiple subjects during the lifetime of the device, and the term “disposable” is used to refer to a device that is intended to be disposed of after use on a single subject.

Embodiments of surgical apparatuses and methods of performing surgical operations will now be described in more detail herein. The following will now describe these surgical apparatuses and methods with reference to the drawings and where like numbers refer to like structures.

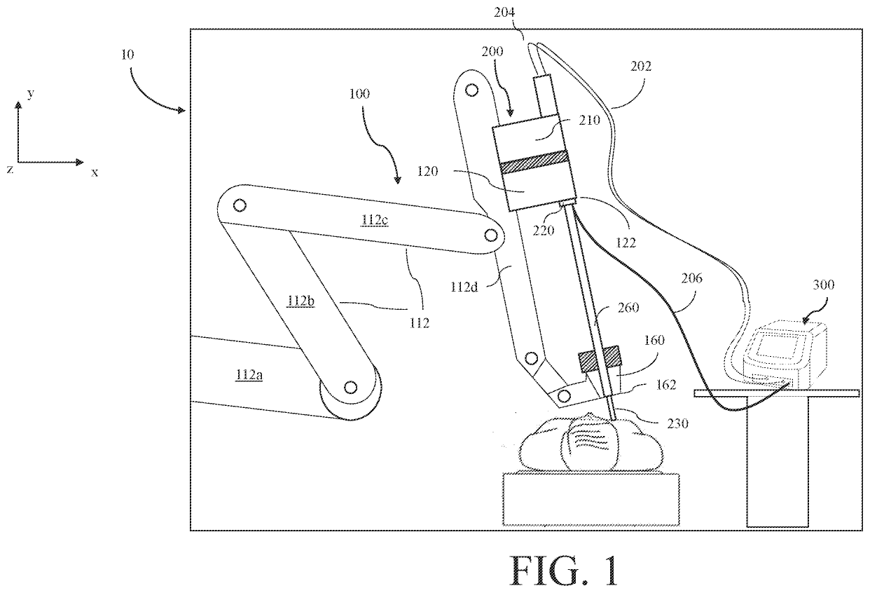

Referring now to FIG. 1, a schematic view of a surgical apparatus 10 is depicted. In these embodiments, the surgical apparatus 10 may include a robotic system 100 and an aspirator 200, such as an ultrasonic aspirator, that may be coupled to the robotic system 100. The ultrasonic aspirator 200 may be particularly suited for ultrasonically fragmenting and aspirating tissue during a surgical operation, as will be described in additional detail herein. Although the surgical apparatus 10 of FIG. 1 is depicted as including the aspirator 200, it should be appreciated that the aspirator is for illustrative purposes only, and the surgical apparatus 10 may include any surgical instrument that may be coupled to and manipulated by the robotic system 100.

As further depicted in FIG. 1, the aspirator 200 may include a handpiece 210, a horn 230, such as an ultrasonic horn, a flue 260 coaxially disposed about the horn 230, and a connector 220 for securing the horn 230 and the flue 260 to the handpiece 210. In these embodiments, the connector 220 may extend from a proximal end 222 of the handpiece 210, and may be used to couple the horn 230 and the flue 260 to the handpiece 210. In some embodiments, the horn 230 and the flue 260 may be directly coupled to the connector 220 (e.g., via threaded engagement, snap-fit, press-fit, etc.). In other embodiments, the horn 230 and the flue 260 may further include tubing and/or fittings that are used to secure the horn 230 and the flue 260 to the connector 220, as will be described in additional detail herein.

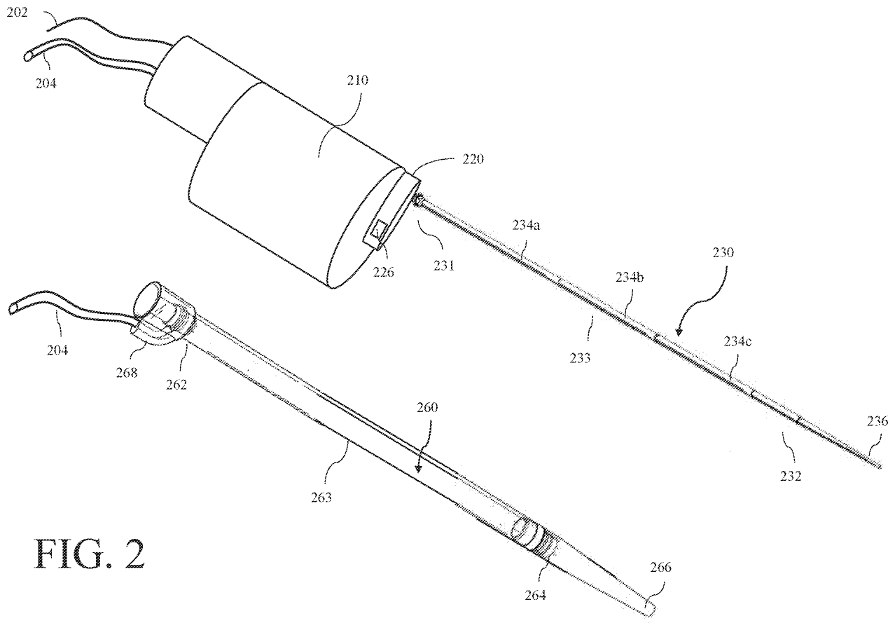

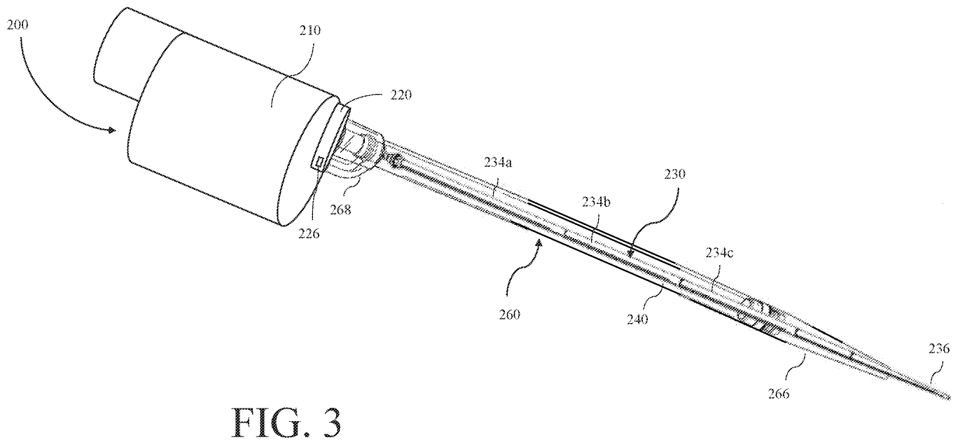

In the embodiments described herein, the handpiece 210, connector 220, horn 230, and flue 260 may be received by the robotic system 100, such that the robotic system 100 is capable of positioning, manipulating, and/or stabilizing the aspirator 200 during a surgical procedure. Once positioned within a target site, the horn 230 may be activated to ultrasonically fragment tissue located at the target site and suction effluent generated during the procedure away from the target site. The aspirator 200 will be described in additional detail herein with reference to FIGS. 2 and 3.

Referring still to FIG. 1, the robotic system 100 may include a plurality of arm members 112, which may be used to manipulate (e.g., move, rotate, etc.) the aspirator 200 through a plurality of planes (e.g., three-dimensional space). For example, the robotic system 100 may include a first arm member 112a, a second arm member 112b, a third arm member 112c, and a fourth arm member 112d, with each of the plurality of arm members 112a-112d being hingedly coupled to each adjacent arm member 112a-112d via a plurality of joints 14, such that the plurality of arm members 112a-112d are capable of extending, retracting, and or rotating about the plurality of joints 14 (e.g., in the x-direction and/or y-direction as depicted in the coordinate axes of FIG. 1). Although not depicted, in other embodiments, it should be understood that the plurality of arm members 112a-112d may be coupled such that each of the arm members 112a-112d are further capable of three-dimensional rotation (e.g., about the x-axis, y-axis, and z-axis as depicted in the coordinate axes of FIG. 1). These embodiments may be particularly advantageous to adjust the angle of the aspirator 200 relative a target site, as will be described in additional detail herein.

Although not depicted, it should be further appreciated that, in some embodiments, the robotic system 100 may further include a base to which at least one of the plurality of arm members 112a-112d is attached. In these embodiments, the base may be a stationary base or a movable base (e.g., movable via wheels, etc.). For example, in embodiments in which the base is a movable base, the robotic system 100 of the surgical apparatus 10 may be moved between locations (e.g., operating rooms), which may allow for the robotic system 100 to be used in connection with multiple surgical apparatuses 10 and/or patients in small period of time. Furthermore, by allowing the robotic system 100 to be operable with a variety of surgical apparatuses, it may be possible to reduce the costs associated with utilizing the robotic system during a particular procedure.

Referring still to FIG. 1, the robotic system 100 may further include a first receiver 120, such as a handpiece receiver, and a second receiver 160, such as a flue receiver, that may be used to couple the aspirator 200 to the robotic system 100. For example, in the embodiments depicted in FIG. 1, at least one of the plurality of arm members 112a-112d may be an alignment arm member (e.g., arm member 112d in FIG. 1), which may include the first receiver 120 and the second receiver 160. Accordingly, when the aspirator 200 is secured to the robotic system 100, the alignment arm member, first receiver 120, and second receiver 160 may align the aspirator 200 in a desired orientation before the aspirator 200 is inserted into the target site. Furthermore, once the aspirator 200 is positioned in the desired orientation, the robotic system 100 may ensure that the aspirator 200 remains axially aligned throughout the course of a surgical operation.

In order to accommodate the aspirator 200, the first receiver 120 may include at least one first receiver port 122, while the second receiver 160 may include at least one second receiver port 162. In these embodiments, the at least one first receiver port 122 and the at least one second receiver port 162 may be axially aligned, such that the aspirator 200 may be linearly inserted through the first receiver 120 and the second receiver 160 simultaneously without need for disassembling the aspirator 200. For example, as depicted in FIG. 1, the handpiece 210 of the aspirator 200 may be inserted into the first receiver 120 such that the connector 220, horn 230, and flue 260 extend through the at least one first receiver port 122 of the first receiver 120.

As further depicted in FIG. 1, with the handpiece 210 positioned within the first receiver 120, the flue 260 may extend into the at least one second receiver port 162 of the second receiver 160. In these embodiments, the flue 260 may include a tapered distal end (as depicted in FIGS. 2 and 3), such that the flue 260 is confined within the at least one second receiver port 162 and does not extend beyond the second receiver 160. The horn 230 may extend through the flue 260 and second receiver 160, such that the horn 230 may be disposed within a target site. Because the at least one first receiver port 122 and the at least one second receiver port 162 are axially aligned, inserting the aspirator 200 through both the first receiver 120 and the second receiver 160 may ensure that the aspirator 200 remains in alignment with the alignment arm member (e.g., arm member 12d as depicted in FIG. 1) as the robotic system 100 is actuated. Accordingly, in these embodiments, the aspirator 200 may be manipulated (e.g., positioned, extended, retracted, rotated, etc.) by manipulating the alignment arm member.

In the embodiments described herein, the handpiece 210 of the aspirator 200 may be inserted into the first receiver 120 such that the handpiece 210 remains secure within the first receiver 120 when the robotic system 100 is actuated. For example, the first receiver 120 may include a plurality of slots that are engaged by a plurality of rails positioned on the handpiece, such that the handpiece 210 may be slidably engaged with the first receiver 120. In other embodiments, the first receiver 120 may include a snap-fit and/or latching mechanism that engages the handpiece 210 when the handpiece 210 is fully inserted into the first receiver 120 (e.g., when the connector 220 extends through the at least one first receiver port 122). In other embodiments still, the first receiver 120 may include a strap, or other similar restraining mechanism, which is secured about a distal end 224 of the handpiece 210 once the handpiece 210 is inserted into the first receiver 120. It should be understood that the examples provided herein are intended to be illustrative in nature, and the handpiece 210 may be secured to the first receiver 120 using any mechanism that effectively secures the handpiece 210 within the first receiver 120 as the robotic system 100 is actuated and/or manipulated.

Although the aspirator 200 is described as being inserted into the first receiver 120 and the second receiver 160 simultaneously, it should be appreciated that the various components of the aspirator 200 may be individually coupled to the robotic system 100. For example, the handpiece 210 may be inserted into the first receiver 120, such that the connector 220 extends from the at least one first receiver port 122 of the first receiver 120. Once the handpiece 210 is secured, the horn 230 and flue 260 may be coupled to the connector 220, at which point the aspirator 200 may be repositioned such that the flue extends into the at least one second receiver port 162, as has been described herein.

Referring still to FIG. 1, the surgical apparatus 10 may further include a console 300, which may be used to control the robotic system 100 and aspirator 200. For example, as depicted in FIG. 1, the surgical apparatus 10 includes a power line 202, an aspiration tube 204, and an irrigation tube 206, which may each be used to connect the aspirator 200 to the console 300. In these embodiments, the power line 202 may supply electrical power to the aspirator 200, while the aspiration tube 204 provides suction and a path for aspiration from the target site to a collection canister (not depicted). The irrigation tube 206 may further provide irrigation fluid through the flue 260 and to the target site during a procedure. Furthermore, although not depicted in FIG. 1, the console 300 may further include a number of control inputs for operating the robotic system, such as manual inputs, foot pedals, and other similar components.

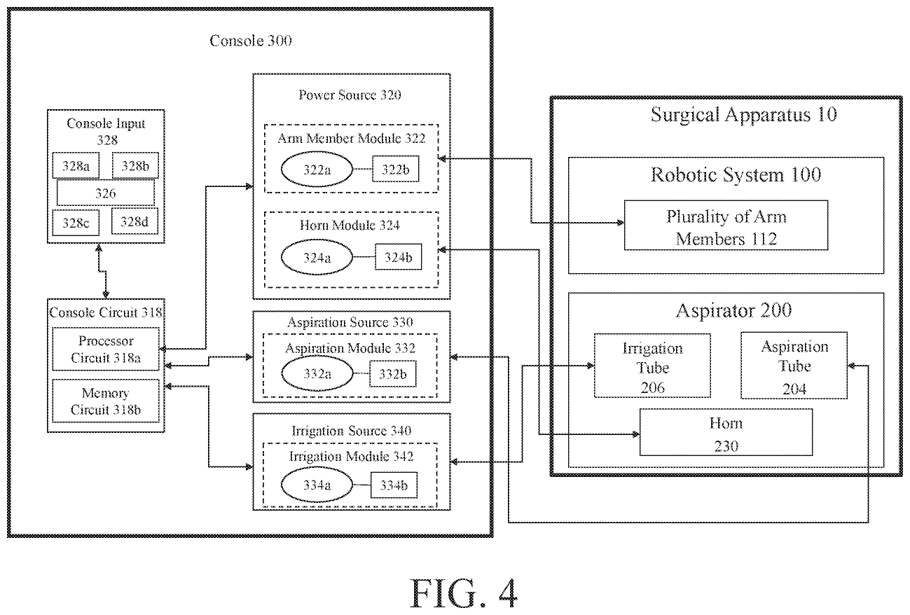

In addition to controlling the aspiration tube 204 and irrigation tube 206 of the surgical apparatus, it should be noted that, in some embodiments, the console 300 may be further configured to control RF energy that is supplied from the handpiece 210 to horn 230 of the aspirator 200. In these embodiments, the RF energy supplied to the horn 230 may be used to aid in coagulation at the target site into which the aspirator 200 is inserted. Furthermore, the handpiece 210 may also include a force sensing element, such as a piezoelectric ceramic force sensing element, that may enable enhanced tissue selectivity within the target site during a surgical procedure. A variety of tissue selectivity mechanisms may be used for ultrasonic aspiration as described in U.S. Pat. No. 10,687,840 to Cotter et al., which is incorporated herein by reference in its entirety. In these embodiments, the tissue selectivity mechanism (e.g., force sensing element, etc.) may be further operated via the console 300. It should be appreciated that, by configuring the console 300 to control the aspiration, irrigation, RF coagulation, and tissue selectivity components and mechanisms of the aspirator 200, the aspirator 200 may be better suited for coupling with the disclosed robotic system 100. The console 300 will be described in additional detail herein with reference to FIG. 4.

Referring now to FIGS. 1-3, the aspirator 200 is depicted in additional detail. As shown most clearly in FIGS. 2 and 3, the handpiece 210 may include a transducer (not depicted) on which the horn 230 (e.g., ultrasonic horn) is fastened. The horn 230 may be powered by the transducer and may be ultrasonically actuated to fragment tissue and suction effluent via aspiration tube 204. In operation, the transducer may convert electrical energy to mechanical energy to transduce energy at ultrasonic frequencies in order to vibrate the horn 230 for fragmenting tissue located at the target site. Furthermore, in these embodiments, the handpiece 210 may further include a connection for radiofrequency (“RF”) energy to support coagulation within the target site, which may occur by conducting a current through the tissue at the target site. It should be further appreciated that the aspiration tube 204 may extend through the handpiece 210, such that the aspiration tube 204 may be directly connected to the horn 230, thereby allowing for effluent generated during the surgical procedure to be removed from the target site via the aspiration tube 204.

Referring still to FIGS. 1-3, the horn 230 has a proximal end 231, a distal end 232, and a body 233 that extends from the proximal end 231 to the distal end 232 and may be defined by a plurality of horn members 234. For example, as depicted in FIGS. 2 and 3, the plurality of horn members 234 may include a first horn member 234a, a second horn member 234b extending distally from the first horn member 234a, and a third horn member 234c extending distally from the second horn member 234b. In these embodiments, the horn members 234a-234c may be coupled/connected via a threaded coupling, or any other similar coupling capable of securing the horn members 234a-234c (e.g., torqueing, laser welding, pins, etc.).

Although the horn 230 is depicted as including three horn members 234 in the embodiments illustrated in FIGS. 2 and 3, it should be appreciated that the horn 230 may include any number of horn members 234 without departing from the scope of the present disclosure. In these embodiments, the plurality of horn members 234 may be used to vary the length (e.g. overall) of the body 233 of the horn 230 for one or more applications. Furthermore, the length (e.g., overall) of the body 233 of the horn 230 may be determined based on the size of the flue 260 used in connection with the horn 230 and/or the dimensions of the robotic system 100 used to control the aspirator 200. For example, in order to effectively secure the aspirator 200 within the robotic system 100, the aspirator 200 may have a length (e.g., overall) that is longer than a length between the first receiver 120 and the second receiver 160, such that the first receiver 120 and the second receiver 160 may stabilize the aspirator 200 during a surgical procedure.

As further depicted in FIGS. 1-3, the horn 230 may further include a surgical tip 236, which may be inserted into a target site such that ultrasonic vibration of the horn 230 is transferred to the target site. In these embodiments, the number of the plurality of horn members 234 included in the horn 230 may be further determined based on the wavelength of the frequency of resonance that is to be supplied to the target site. For example, in some embodiments, the horn 230 may be activated to vibrate in the ultrasonic frequency range with a longitudinal amplitude between about 5 millimeters (0.005 meter) and 14 millimeters (0.014 meter).

In these embodiments, the surgical tip 236 may have an open end and may be coupled to the distal most horn member 234 of the plurality of horn members 234. Accordingly, when the aspirator 200 is inserted into the target site and vacuum is applied to the aspiration tube, effluent may be drawn into the aspirator via the surgical tip 236 of the horn 230 and transported away from the target site.

Referring still to FIGS. 1-3, the horn 230 may be substantially circular in cross-section and may be coaxially disposed within the flue 260 (e.g., as depicted in FIG. 3). In these embodiments, the flue 260 and the horn 230 may define an annular cavity 240 therebetween, such that an irrigation fluid may be supplied through the annular cavity 240 and into the target site via the surgical tip 236, as will be described in additional detail herein.

The flue 260 may include a proximal end 262, a distal end 264, and a body 263 that extends between the distal end 264 of the flue 260 and the proximal end 262 of the flue. The distal end 264 may further include a flue tip 266, which may be inserted into the second receiver 160 of the robotic system 100. In these embodiments, the surgical tip 236 positioned on the horn 230 may extend through the flue tip 266 and the second receiver 160 when the aspirator 200 is coupled to the robotic system 100, such that the surgical tip 236 may be inserted into the target site.

In these embodiments, the flue 260 may be configured to deliver irrigation fluid to the target site and to further isolate the ultrasonically activated horn 230 and surgical tip 236 when the horn 230 is inserted into the target site. Additionally, the flue 260 may provide a dielectric boundary between the energized horn 230 and tissues positioned outside of the target site. In order to provide the dielectric boundary between the horn 230 and tissues positioned outside of the target site, in some embodiments, the flue tip 266 may be made of silicone or any other similar material capable of forming the boundary. Furthermore, the body 263 may be formed of a rigid material (e.g., a material having rigidity of between approximately 100,000 psi-350,000 psi) to ensure that the horn 230 and aspirator 200 remain stabilized when the horn 230 is activated.

Referring again to FIGS. 1-3, the aspirator 200 may further include a cooling and irrigation system that may provide cooling fluid and irrigation fluid to the horn 230 to maintain the temperature of the horn 230 within an acceptable range and clear effluent from the target site. In these embodiments, the irrigation tube 206 of the aspirator 200 may be coupled to a flue tube 268 in order to provide irrigation fluid, such as saline, through the annular cavity 240 defined between the flue 260 and the horn 230. As most clearly depicted in FIG. 1, the irrigation tube 206 may be coupled to the console 300, which may be configured for supplying the irrigation fluid. For example, the console 300 may include a pump, such as a peristaltic pump, that may provide irrigation fluid to the flue 260 at a particular pump rate. In some embodiments, the peristaltic pump rate may be as a low as 2-3 mL/minute.

As further depicted in FIGS. 1-3, the irrigation fluid supplied to the target site may be removed via the aspiration tube 204, such that a desired amount of irrigation fluid is available at the target site in a continuous fluid circuit. In these embodiments, the irrigation fluid may aid in preventing and/or reducing immediate clotting of blood that may clog the surgical tip 236 and/or aspiration tube 204. Furthermore, the irrigation fluid that pools near the surgical tip 236 (e.g., prior to being removed via the aspiration tube 204) may be used to cool the surgical tip 236 and the target site.

Although the irrigation fluid supplied to the target site may provide the benefits described herein, in some embodiments, the capillarity of the irrigation fluid may cause the irrigation fluid to pool, or “dam,” at the surgical tip 236. In these embodiments, the damming of the irrigation fluid may cause the aspirator 200 and/or the annular cavity 240 to at least partially clog, which may reduce the continuous circuit of irrigation described herein. This issue may be exacerbated in embodiments in which the diameter of the annular cavity 240 is reduced, either due to the diameter of the flue 260, the horn 230, or both.

As depicted most clearly in FIGS. 2 and 3, the pooling of irrigation fluid near the surgical tip 236 may be alleviated by providing a venting mechanism, such as a vent 226, between the irrigation tube 206 and the console 300 (e.g., having the pump or other similar mechanism that provides the irrigation fluid to the aspirator). In these embodiments, the vent 226 may be an atmospheric vent that exposes the annular cavity 240 to atmosphere, which may in turn alleviate pressure from around the surgical tip 236 that may cause the irrigation fluid to pool. For example, in the embodiments described herein, the pumping of irrigation fluid into the aspirator 200 may result in a small vacuum being formed near the surgical tip 236 of the aspirator 200. However, by exposing the annular cavity 240 to atmosphere via the vent 226, it may be possible to ensure that pressure within the aspirator 200 is released to an external atmosphere, which may thereby prevent the pooling of irrigation fluid in the aspirator 200.

As depicted in FIGS. 2 and 3, the vent 226 is disposed on the connector 220 of the handpiece 210. In these embodiments, the irrigation fluid may flow from the console 300 through the irrigation tube 206 and into the flue tube 268, at which point the irrigation fluid may be deposited in the annular cavity 240 of the aspirator 200. Because the flue tube 268 may be fluidly coupled to the connector 220, providing the vent 226 within a portion of the connector 220 may further act to ventilate the annular cavity 240 of the aspirator 200, as has been described herein. Although the vent 226 is depicted as being formed within the connector 220, it should be appreciated that the vent 226 may be positioned at any point between the console 300 and the flue 260 such that the vent 226 may expose the annular cavity 240 to atmosphere.

Turning now to FIG. 4, a schematic view of the console 300 is depicted. As will be described in additional detail herein, the console 300 may be configured to control operation of both the robotic system 100 (e.g., the orientation and/or position of the plurality of arm members 112a-112d, and in turn, the aspirator 200) and the aspirator 200 (e.g., activation of the horn 230, aspiration tube 204, and/or irrigation tube 206). As will be appreciated in view of the foregoing, an individual user (e.g., surgeon or other technician) may be capable of performing a MIS, such as a laparoscopy, without assistance by manipulating the console to control the various components of the robotic system 100 and aspirator 200.

As further depicted in FIG. 4, the console 300 may include a console circuit 318, a power source 320, an aspiration source 330, and an irrigation source 340. The console 300 may further include a console input 328, such as a computer, which may allow a user to operate the console 300. In these embodiments, the console input 328 may be configured to provide automatic control of the console 300 via a software program, or may be manually controlled via a user manipulating a user interface and/or control mechanisms thereof.

Referring still to FIG. 4, the console input 328 may include a user interface (e.g., control buttons and visual/aural indicators, such as a display and/or speakers, with the control buttons providing user control over various functions of the console 300, and with the visual/aural indicators providing visual/aural feedback of the status of one or more conditions and/or positions of components of the console 300). The control buttons may include one or more buttons for adjusting the position and/or orientation of each of the plurality of arm members 112 of the robotic system 100, activating the horn 230 and controlling the frequency at which the horn 230 vibrates, activating a vacuum within the aspiration tube 204, and/or supplying irrigation fluid to the aspirator via the irrigation tube 206. actuating the mechanism(s) For example, the console input 328 may include one or more buttons and/or knobs 328a, 328b for adjusting the position and orientation of each of the plurality of arm members 112, and one or more buttons (e.g., a foot pedal) and/or knobs 328c, 328d for activating the horn 230, the aspiration source 330, the irrigation source 340, and/or adjusting the control parameters of any of these components.

Furthermore, it should be appreciated that, in some embodiments, multiple buttons and/or knobs of the console input 328 may be used to control multiple components of the surgical apparatus. For example, in some embodiments, the console input 328 may include a foot pedal, which may include one or more buttons and/or knobs for controlling the irrigation source 340 and the aspiration source 330, as will be described in additional detail herein.

Referring still to FIG. 4, in these embodiments, console circuit 318 is electrically and communicatively coupled to power source 320, aspiration source 330, and irrigation source 340, such as by one or more wires or circuit traces. Console circuit 318 may be assembled on an electrical circuit and may include, for example, a processor circuit 318a and a memory circuit 318b.

Processor circuit 318a has one or more programmable microprocessors and associated circuitry, such as an input/output interface, buffers, memory, etc. Memory circuit 318b is communicatively coupled to processor circuit 318a, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), flash memory, etc. Console circuit 118 may be formed as one or more Application Specific Integrated Circuits (ASIC).

Console circuit 318 is configured via software and/or firmware residing in memory circuit 318b to execute program instructions to perform functions associated with the surgical apparatus 10. For example, the console circuit 318 may adjust the position and/or orientation of each of the plurality of arm members 112 of the robotic system, activate the horn 230, aspiration source 330, and/or irrigation source 340, adjust the frequency at which the horn 230 vibrates, and/or adjust the volume of irrigation fluid that is supplied to the irrigation tube 206.

Referring still to FIG. 4, the power source 320 may include, for example, an arm member module 322 and a horn module 324. In these embodiments, each of the arm member module 322 and the horn module 324 may be electrically and controllably coupled to the console circuit 318. As provided herein, the arm member module 322 and the horn module 324 may be electrically coupled to the console circuit 318 by way of electrical wiring or any other suitable electrical connection, such that user inputs on the console input 328 may be relayed to the console circuit 318 and used to control the power delivered by the source 320 to the arm member module 322 and the horn module 324.

In these embodiments, the arm member module 322 may include a power supply 322a, to which an electric lead 322b is attached. For example, the power supply 322a may be connected to each of the plurality of arm members 112 such that the power supply 322a may be used to adjust the position and orientation of each of the plurality of arm members 112. Similarly, the horn module 324 may include a power supply, such as a generator 324a, to which an electric lead 324b is attached. In these embodiments, the generator may be utilized to control the power supplied to the horn 230, such that the horn module 324 is able to control the frequency at which the horn 230 vibrates once the horn 230 is activated.

Referring still to FIG. 4, aspiration source 330 may include an aspiration module 332. The aspiration module may include, for example, a power supply 332a that may be used to activate a vacuum assembly (not depicted) and generate suction within aspiration tube 204 via an electric lead 332b. In these embodiments, the power supply 332a may be further utilized to vary the strength of the suction generated within the aspiration tube 204 in order to meet the aspiration needs of a particular surgical procedure.

As noted herein, the console 300 may further include an irrigation source 340. In these embodiments, the irrigation source 340 may include an irrigation module 342, which may be controlled via the console to supply irrigation fluid to the aspirator 200. The irrigation source 340 may include a fluid supply 334a that may be coupled to the irrigation tube 206 via a fluid coupling 334b, such that irrigation fluid may pass from the console 300 to the aspirator 200 when the irrigation source 340 is activated.

Referring still to FIG. 4, in the embodiments described herein, the console 300 may be electrically and operably connected to robotic system 100 and aspirator 200 such that the console 300 is capable of controlling and/or manipulating the robotic system 100 and aspirator 200 simultaneously during a surgical procedure. For example, once the aspirator 200 is coupled to the robotic system, the arm member module 322 may be activated to adjust the position and/or orientation of the plurality of arm members 112, and in turn, the aspirator 200, such that the aspirator is positioned at an entry position and an entry orientation. It should be appreciated that, depending on the surgical procedure being performed, the aspirator 200 may be positioned at various locations and at various angles relative to a target site. For example, during some laparoscopic procedures, the surgical tip of the horn 230 may be angled at approximately 30-45 degrees relative the target site prior to being inserted into the target site.

Once the arm member module 322 has positioned the aspirator 200 at a particular position and angle relative the target site, the arm member module 322 may be further controlled to insert the aspirator into the target site at the entry position and entry orientation. Furthermore, it should be understood that, in these embodiments, the arm member module 322 may be deactivated, such that the aspirator 200 remains stabilized in the desired position and orientation during the surgical procedure. In the event the aspirator 200 requires adjustment during the procedure, the arm member module 322 may be reactivated in order to reposition and/or rotate the aspirator 200.

With the aspirator 200 positioned in the target site, the console 300 may be used to activate the horn module 324, which may in turn provide power to the horn via 230 the generator 324a. In these embodiments, a user may adjust the power delivered to the horn 230 in order to control the frequency at which the horn 230 vibrates.

As the horn 230 vibrates, the console 300 may be further utilized to operate the aspiration source 330 and the irrigation source 340. For example, and as has been described herein, the aspiration source 330 and the irrigation source 340 may be controlled via a common control mechanism, such as a foot pedal. Accordingly, each of the aspiration module 330 and irrigation source 340 may be activated individually and as needed throughout the surgical procedure in order to aspirate effluent from the target site and/or provide irrigation fluid to the target site.

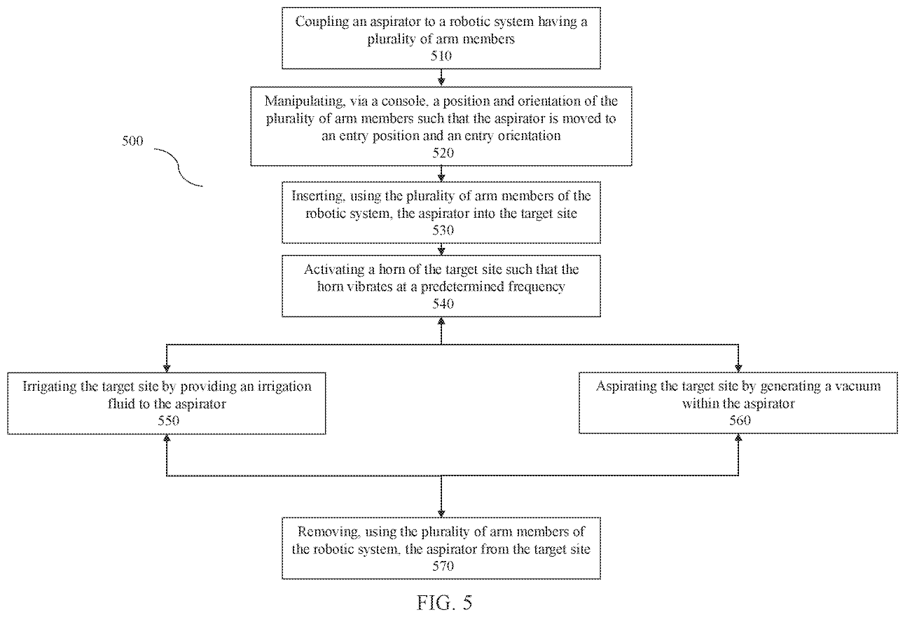

Turning now to FIG. 5, an illustrative method 500 of performing a surgical operation is depicted. In the embodiments described herein, the method 500 may be particularly suited for providing aspiration during a MIS, such as a laparoscopy. However, it should be understood that the embodiments described herein are for illustrative purposes only, and the method 500 may be used for performing any surgical operation without departing from the scope of the present disclosure.

As depicted at block 510, the method 500 may initially involve coupling a surgical instrument, such as an aspirator, to a robotic system having a plurality of arm members. Once the surgical instrument is coupled to the plurality of arm members, the method may advance to block 520, which may involve manipulating the position and orientation of the plurality of arm members, using a console, such that the aspirator is moved to a desired position and orientation relative a target site.

With the aspirator moved to the desired position and orientation, the method may move to block 530, which may involve inserting, using the plurality of arm members of the robotic system, the aspirator into the target site. As the aspirator enters the target site, a horn of the aspirator may be activated such that the horn vibrates at a predetermined frequency within the target site and fragments tissue at the target site, as depicted at block 540.

As the horn vibrates, a user may simultaneously provide an irrigation fluid to the target site and aspirate the target site, as is depicted at block 550 and 560, respectively. In these embodiments, the console may include an irrigation source and an aspiration source that may be individually controlled via a single control input, such as a foot pedal, that allows a user to selectively operate each of the irrigation source and the aspiration source during the course of the surgical procedure. The irrigation source and aspiration source may be selectively operated as needed until the surgical procedure is complete. Once the procedure is complete, the method may move to block 570, which may include removing the aspirator from the target site, using the plurality of arm members of the robotic system.

As should be appreciated in view of the foregoing, a surgical apparatus is disclosed herein. The surgical apparatus may include a surgical device, such as an aspirator, and a robotic system that may be used to manipulate the surgical device. The surgical apparatus may further include a console, which may be configured to independently control operation of both the robotic system and the surgical device. In these embodiments, a user performing the procedure may be capable of controlling the robotic system throughout the course of the procedure (e.g., via foot pedal, etc.) such that the surgical procedure may be performed by a single user.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A surgical apparatus comprising:
a robotic system comprising:
a plurality of arm members, each of the plurality of arm members being coupled to each adjacent arm member via a joint, and at least one of the plurality of arm members including a first receiver and a second receiver;
an aspirator comprising:
a handpiece;
a connector extending from the handpiece;
a horn coupled to the handpiece via the connector; and
a flue coaxially disposed about the horn and coupled to the handpiece via the connector;
wherein the handpiece of the aspirator is received by the first receiver of the at least one of the plurality of arm members and the flue of the aspirator is received by the second receiver of the at least one of the plurality of arm members, such that the aspirator remains axially aligned relative to the at least one of the plurality of arm members when the robotic system is manipulated.

2. The surgical apparatus of claim 1, wherein the flue further comprises a distal end and a proximal end, and the distal end of the flue is received by the second receiver.

3. The surgical apparatus of claim 2, wherein the horn further includes a distal end and a proximal end, and the distal end of the horn extends beyond the distal end of the flue and the second receiver when the flue is received by the second receiver.

4. The surgical apparatus of claim 3, wherein the handpiece further comprises a transducer that vibrates the horn at a predetermined frequency when the horn is coupled to the connector of the handpiece.

5. The surgical apparatus of claim 1, further comprising a console for individually controlling the robotic system and the aspirator.

6. The surgical apparatus of claim 5, further comprising an aspiration tube for providing a vacuum to the horn, wherein the aspiration tube extends between the handpiece of the aspirator and the console.

7. The surgical apparatus of claim 6, further comprising an annular cavity defined by a space between the horn and the flue when the flue is coaxially disposed about the horn.

8. The surgical apparatus of claim 7, further comprising an irrigation tube for supplying irrigation fluid to the annular cavity, wherein the irrigation tube extends between the aspirator and the console.

9. The surgical apparatus of claim 8, wherein the connector of the handpiece further comprises a vent for ventilating the irrigation tube to atmosphere.

10. The surgical apparatus of claim 9, wherein the console is configured to selectively control supplying the irrigation fluid to the annular cavity and providing the vacuum to the horn via a foot pedal.

11. The surgical apparatus of claim 1, wherein each of the plurality of arm members are coupled to each adjacent arm member such that each of the plurality of arm members are capable of three-dimensional movement.

12. A surgical apparatus comprising:
a robotic system comprising:
a plurality of arm members, each of the plurality of arm members being coupled to each adjacent arm member via a joint, and at least one of the plurality of arm members including a first receiver and a second receiver;
an aspirator comprising:
a handpiece;
a connector extending from the handpiece, the handpiece being received by the first receiver such that the connector extends from the first receiver;
a horn coupled to the handpiece via the connector, the horn having a distal end, a proximal end, and a body extending therebetween; and
a flue coaxially disposed about the horn and coupled to the handpiece via the connector, the flue having a distal end, a proximal end, and a body extending therebetween, the distal end of the flue being received by the second receiver such that the distal end of the horn extends beyond the distal end of the flue and the second receiver; and
a console comprising:
an irrigation source that provides irrigation fluid to the aspirator via an irrigation tube;
an aspiration source that provides suction to the aspirator via an aspiration tube; and
a power source that provides power to the aspirator and the plurality of arm members;
wherein the connector further includes a vent for ventilating the irrigation tube to atmosphere when the irrigation source provides the irrigation fluid to the aspirator.

13. The surgical apparatus of claim 12, wherein the console is configured to selectively control supplying the irrigation fluid to the aspirator and providing the suction to the aspirator via a foot pedal.

14. The surgical apparatus of claim 12, wherein the handpiece further comprises a transducer that vibrates the horn at a predetermined frequency when the horn is coupled to the connector of the handpiece.

15. The surgical apparatus of claim 12, wherein each of the plurality of arm members are coupled to each adjacent arm member such that each of the plurality of arm members are capable of three-dimensional movement.

16. The surgical apparatus of claim 12, wherein the aspirator remains axially aligned relative to the at least one of the plurality of arm members when the robotic system is manipulated.

17. A method of performing a surgical operation comprising:
coupling an aspirator to a robotic system having a plurality of arm members, wherein at least one of the plurality of arm members includes a first receiver and a second receiver for receiving the aspirator;
manipulating, via a console, a position and orientation of the plurality of arm members, such that the aspirator is moved to an entry position and an entry orientation relative a target site;
inserting, using the plurality of arm members of the robotic system, the aspirator into the target site at the entry position and the entry orientation;
activating, using the console, a horn of the aspirator such that the horn vibrates within the target site at a predetermined frequency;
irrigating, via the console, the target site by supplying an irrigation fluid through an irrigation tube that extends from the console to the aspirator;
aspirating, via the console, the target site by generating a vacuum within the aspirator using an aspiration tube that extends from the console to the aspirator; and
removing, using the plurality of arm members of the robotic system, the aspirator from the target site.

18. The method of claim 17, wherein the method steps of irrigating the target site and aspirating the target site may be selectively controlled by the console using a foot pedal.

19. The method of claim 17, wherein each of the plurality of arm members are coupled to each adjacent arm member such that each of the plurality of arm members are capable of three-dimensional movement.

20. The method of claim 17, wherein the entry orientation of the aspirator is at an angle of 30-45 degrees relative to the target site.
